(19) United States
(12) Patent Application Publication
(10) Pub. No.: US 2023/0067079 A1
(43) Pub. Date: Mar. 2, 2023

(54) IN-SITU RUBBER MATRIXES FOR ELASTIC AND PHOTO-PATTERNABLE POLYMER SEMICONDUCTORS AND DIELECTRICS

(71) Applicant: The Board of Trustees of the Leland Stanford Junior University, Stanford, CA (US)

(72) Inventors: Yu ZHENG, Stanford, CA (US); Zhenan BAO, Stanford, CA (US); Zhiao YU, Stanford, CA (US)

(73) Assignee: The Board of Trustees of the Leland Stanford Junior University, Stanford, CA (US)

(21) Appl. No.: 17/899,512

(22) Filed: Aug. 30, 2022

Related U.S. Application Data
(60) Provisional application No. 63/238,723, filed on Aug. 30, 2021.

(51) Int. Cl. C08L 9/00

(52) U.S. Cl. CPC C08L 9/00

(57) ABSTRACT

Next-generation wearable electronics require enhanced mechanical robustness and device complexity. Besides previously reported softness and stretchability, desired merits for practical use include elasticity, solvent resistance, photo-patternability and high charge carrier mobility. The present embodiments provide a molecular design concept that simultaneously achieves all these targeted properties in both polymeric semiconductors and dielectrics, without compromising electrical performance. This is enabled by covalently-embedded in-situ rubber matrix (iRUM) formation through good mixing of iRUM precursors with polymer electronic materials, and finely-controlled composite film morphology built on azide crosslinking chemistry which leverages different reactivities with C—H and C═C bonds. The high covalent crosslinking density results in both superior elasticity and solvent resistance. When applied in stretchable transistors, the iRUM-semiconductor film retained its mobility after stretching to 100% strain, and exhibited record-high mobility retention of 1 cm² V⁻¹ s⁻¹ after 1000 stretching-releasing cycles at 50% strain. The cycling life was stably extended to 5000 cycles, five times longer than all reported semiconductors.

### CROSS-REFERENCE TO RELATED APPLICATIONS

The present application claims priority to U.S. Provisional Patent Application No. 63/238,723 filed Aug. 30, 2021, the contents of which are incorporated herein by reference in their entirety.

### STATEMENT OF GOVERNMENT SUPPORT AND/OR SPONSORSHIP

This invention was made with Government support under contract FA9550-18-1-0143 awarded by the Air Force Office of Scientific Research. The Government has certain rights in the invention.

### TECHNICAL FIELD

The present embodiments relate generally to wearable electronics having high electrical performance combined with elasticity, solvent resistance, and facile patternability, and more particularly to in-situ rubber matrixes for elastic and photo-patternable polymer semiconductors and dielectrics.

### BACKGROUND

Skin-like electronics have garnered considerable interests over the past decade for their potential applications in robotics, prosthetics, health monitoring and medical implants. Currently, stretchable electronics are made by either applying geometric designs on rigid inorganic-based devices using buckled substrates or developing intrinsically stretchable organic electronic materials. Nevertheless, softness and stretchability reported before are still far from the requirements for realistic consumer electronics. High electrical performance combined with elasticity, solvent resistance, and facile patternability are desired merits from both daily-use and practical manufacturing perspectives. However, each property usually requires a particular molecular design and has not been possible concurrently.

It was against this technological backdrop that the present Applicant south a technological solution to these and other problems deeply rooted in this technology.

### SUMMARY

The present embodiments relate to next-generation wearable electronics with enhanced mechanical robustness and complexity, in addition to high mobility, elasticity, solvent resistance and photo-patternability. In some embodiments, a molecular design concept simultaneously achieves all these targeted properties in both polymeric semiconductors and dielectrics, without comprising electrical performance. This is enabled by a covalently-embedded in-situ rubber matrix (iRUM) formation designed through excellent miscibility of iRUM precursors with both conjugated and insulating polymers, along with finely controlled composite film morphology built on azide chemistry. The high covalent crosslinking density results in remarkable elasticity and solvent resistance. When applied in fully stretchable transistors, the iRUM-semiconductor film retains its mobility after stretching to 100% strain, and exhibits 1 cm² s′ record-high mobility retention after 1000 stretching-releasing cycles at 50% strain. The cycling life is stably extended to 5000 cycles, five times longer than all previous attempts. These and other embodiments can be fabricated in a fully-patterned elastic transistor array via consecutively photo-patterning of dielectrics and semiconductors, demonstrating the feasibility of integrated solution-processed electronics manufacturing.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIGS. 1a-1d provide a schematic illustration of an example iRUM approach for both semiconductors and dielectrics according to embodiments.

FIGS. 2a-2m illustrate an example systematic investigation of molecular design principles of iRUM semiconductors according to embodiments.

FIGS. 3a-3k illustrate an example characterization of the electrical performance of iRUM-s under mechanical deformation and solvent resistance according to embodiments.

FIGS. 4a-4j illustrate example aspects of the photo-patternability of iRUM-s and iRUM-d, and the integrated patterned elastic transistors on a single substrate according to embodiments.

FIGS. 5a-5d illustrate example generic aspects of a molecular design concept according to embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

### Introduction

Skin-like electronics have garnered considerable interest over the past decade for their potential applications in robotics, prosthetics, health monitoring and medical implants (Yang, J. C. et al. Electronic Skin: Recent Progress and Future Prospects for Skin-Attachable Devices for Health Monitoring, Robotics, and Prosthetics. Adv. Mater. 31, 1904765 (2019)). Currently, stretchable electronics are made by either applying geometric designs on rigid inorganic-based devices using buckled substrates (Rogers, J. A., Someya, T. & Huang, Y. Materials and Mechanics for Stretchable Electronics. Science (80-.). 327, 1603-1607 (2010); Kaltenbrunner, M. et al. An ultra-lightweight design for imperceptible plastic electronics. Nature 499, 458-463 (2013)) or developing stretchable organic electronic materials through molecular-level design (Lipomi, D. J. & Bao, Z. Stretchable and ultraflexible organic electronics. MRS Bull. 42, 93-97 (2017); Bao, Z. Skin-inspired organic electronic materials and devices. MRS Bull. 41, 897-904 (2016)). Nevertheless, softness and stretchability reported before are still far from the requirements for realistic consumer electronics (Wang, S., Oh, J. Y., Xu, J., Tran, H. & Bao, Z. Skin-Inspired Electronics: An Emerging Paradigm. Acc. Chem. Res. 51, 1033-1045 (2018)). Besides, high electrical performance combined with elasticity, solvent resistance, and facile patternability are desired merits from both daily-use and practical manufacturing perspectives; however, each property usually requires a particular molecular design and has not been possible concurrently.

Polymer electronic materials have been identified as promising candidates due to their potential mechanical flexibility, structural tunability, solution processability and cost-effectiveness (Zhao, Y. et al. Melt-Processing of Complementary Semiconducting Polymer Blends for High Performance Organic Transistors. Adv. Mater. 29, 1605056 (2017); Sirringhaus, H. 25th anniversary article: Organic field-effect transistors: The path beyond amorphous silicon. Adv. Mater. 26, 1319-1335 (2014)). Albeit theoretically promising, the key components, i.e. semiconductors and dielectrics, still face challenges. First, many previously reported polymer electronic materials are either brittle or viscoelastic, which causes unreliable hysteretic performance when subjected to harsh mechanical challenges, e.g. multiple, high-loading stretching-releasing cycles (Kim, J. H. et al. Understanding mechanical behavior and reliability of organic electronic materials. MRS Bull. 42, 115-123 (2017)), making these materials impossible for realistic long-term use. Second, for integrated circuits, sensor array and display fabrication, the semiconductors and dielectrics need to be prepared through a simple yet universal solution-based deposition method, and should be compatible with multilayer device fabrication. However, solution-based processing is usually accompanied by poor solvent resistance. This is thus a ‘mixed blessing’ for polymer electronics, since it results in good solution processability but suffers from chemical dissolution in later processing steps, limiting the possibility of low-cost and scalable production (Freudenberg, J., Jansch, D., Hinkel, F. & Bunz, U. H. F. Immobilization Strategies for Organic Semiconducting Conjugated Polymers. Chem. Rev. 118, 5598-5689 (2018)). Third, facile patternability is another critical merit for polymer electronic materials. Previous patterning methods include protection-etching that requires multiple steps involving sacrificial layer and orthogonal chemicals (Wang, S. et al. Skin electronics from scalable fabrication of an intrinsically stretchable transistor array. Nature 555, 83-88 (2018)), and inkjet printing that has difficulties in producing uniform devices (Liu, J. et al. Fully stretchable active-matrix organic light-emitting electrochemical cell array. Nat. Commun. 11, 3362 (2020)).

To overcome mechanical challenges, developing a stretchable and even elastic (reversible under cyclic loading-unloading) semiconductor without sacrificing electrical performance is a desired yet difficult objective (Root, S. E., Savagatrup, S., Printz, A. D., Rodriquez, D. & Lipomi, D. J. Mechanical Properties of Organic Semiconductors for Stretchable, Highly Flexible, and Mechanically Robust Electronics. Chem. Rev. 117, 6467-6499 (2017)). Most of the conjugated polymers experience device failure under low strain (<10%) due to their semi-crystalline thin film morphology (Xie, R., Colby, R. H. & Gomez, E. D. Connecting the Mechanical and Conductive Properties of Conjugated Polymers. Adv. Electron. Mater. 4, 1700356 (2018)). Virtually all previously reported molecular design rules focused on enhancing the ultimate fracture strains of polymer semiconductors without loss of electronic functionalities, in which ‘reducing long-range crystalline order’ has been a general principle (Ashizawa, M., Zheng, Y., Tran, H. & Bao, Z. Intrinsically stretchable conjugated polymer semiconductors in field effect transistors. Prog. Polym. Sci. 100, 101181 (2020)). However, the semiconductor films in these cases suffered from permanent plastic deformation prior to crack formation. Upon removal of strain, the semiconductor film supported on an elastic dielectric tends to form wrinkles or buckles. Unfortunately, these processes invariably lead to interfacial delamination and degrade charge transport (Zheng, Y. et al. An Intrinsically Stretchable High-Performance Polymer Semiconductor with Low Crystallinity. Adv. Funct. Mater. 29, 1905340 (2019)). For practical applications, the semiconductor needs to function after multiple strain cycles, and thus elasticity/reversibility beyond simple stretchability while maintaining high charge transporting ability is needed. This requires high crosslinking density as well as chain flexibility in the active layer (Heinrich, G., Straube, E. & Helmis, G. Rubber elasticity of polymer networks: Theories. in Polymer Physics 33-87 (Springer Berlin Heidelberg, 1988)), while balancing charge transport over multiple length scales.

To achieve chemical robustness as well as facile patternability, the present Applicant recognizes that photo-patterning using crosslinking chemistry is a viable approach, which generates covalent crosslinking in polymer electronic materials (Kim, M. J. et al. Universal three-dimensional crosslinker for all-photopatterned electronics. Nat. Commun. 11, 1520 (2020); Kwon, H. J. et al. Facile Photo-cross-linking System for Polymeric Gate Dielectric Materials toward Solution-Processed Organic Field-Effect Transistors: Role of a Cross-linker in Various Polymer Types. ACS Appl. Mater. Interfaces 12, 30600-30615 (2020)). However, introducing crosslinkers usually results in higher elastic modulus and lower fracture strain than the pristine semiconductors and dielectrics. Limited examples of crosslinkers have been observed to increase the fracture strain of semiconductors; unfortunately, they all dramatically degraded the charge carrier mobility at a mere 5 w.t.% addition due to the disruption of semiconductor aggregation. These crosslinked semiconductors still exhibited plastic deformation, i.e. irreversibly stretchability, due to limited crosslinking density (Wang, G.-J. N. et al. Tuning the Cross-Linker Crystallinity of a Stretchable Polymer Semiconductor. Chem. Mater. 31, 6465-6475 (2019); Wang, G.-J. N. et al. Inducing Elasticity through Oligo-Siloxane Crosslinks for Intrinsically Stretchable Semiconducting Polymers. Adv. Funct. Mater. 26, 7254-7262 (2016)). Another reported approach was blending a polymer semiconductor with a non-crosslinked elastomeric insulating polymer (Xu, J. et al. Highly stretchable polymer semiconductor films through the nanoconfinement effect. Science (80-). 355, 59-64 (2017); Zhang, G. et al. Versatile Interpenetrating Polymer Network Approach to Robust Stretchable Electronic Devices. Chem. Mater. 29, 7645-7652 (2017); Guan, Y.-S. et al. Air/water interfacial assembled rubbery semiconducting nanofilm for fully rubbery integrated electronics. Sci. Adv. 6, eabb3656 (2020); Zhang, S. et al. Tacky Elastomers to Enable Tear-Resistant and Autonomous Self-Healing Semiconductor Composites. Adv. Funct. Mater. 30, 2000663 (2020)). However, the high percentage of the insulating polymer in the blend rendered the film more susceptible to dissolution from organic solvents than neat semiconductor. In addition, most of the blended insulating polymers are still viscoelastic rather than elastic/reversible. Due to the absence of covalent crosslinking sites, the long-term mechanical stability remains uncertain. For dielectrics, some fluorinated elastomers have been reported to be organic solvent resistant, but they either showed double-layer capacitive effect (Kong, D. et al. Capacitance Characterization of Elastomeric Dielectrics for Applications in Intrinsically Stretchable Thin Film Transistors. Adv. Funct. Mater. 26, 4680-4686 (2016)), or relied heavily on surface-modification to achieve suitable wettability for semiconductor solution deposition.

Overall, no general molecular design approach so far can simultaneously achieve elasticity, solvent resistance and photo-patternability without degrading electrical performance.

iRum Approach

According to certain general aspects, the present embodiments relate to rationally designed single precursors for covalently-embedded in-situ rubber matrix (iRUM) formation, which can undergo both self-crosslinking and crosslinking with the corresponding electronic material in finely controlled ratio, to achieve all targeted properties for both polymeric semiconductors (PSC) and dielectrics.

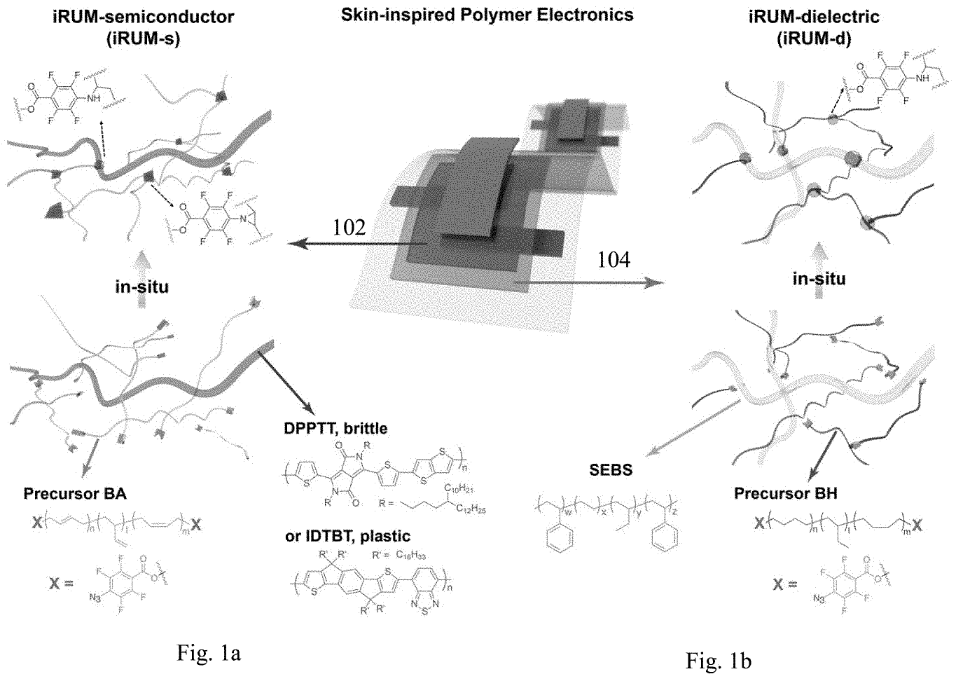

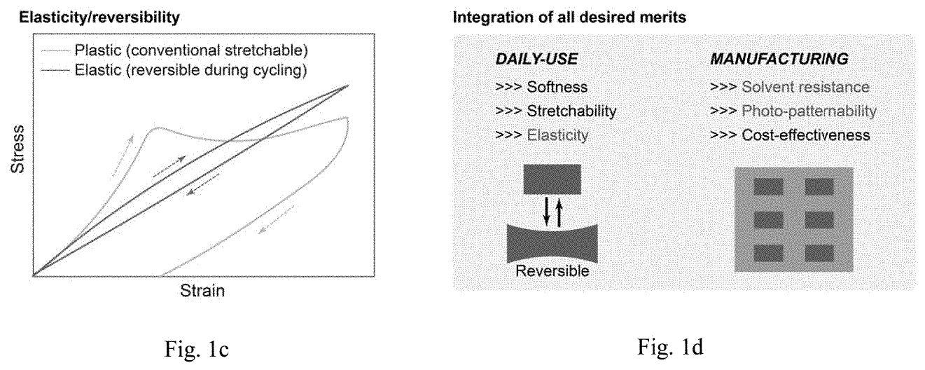

FIGS. 1a-1d provide an example schematic illustration of an iRUM approach for both semiconductors and dielectrics according to embodiments. As shown in FIG. 1a, for semiconductors 102, a DPPTT network is covalently embedded into the in-situ formed elastic rubber matrix generated by iRUM precursor BA. In iRUM-s, the number of covalent crosslinking sites created through azide/C═C cycloaddition is much higher than that created through azide/C—H insertion. FIG. 1b illustrates that for dielectrics 104, SEBS is uniformly crosslinked by iRUM precursor BH through azide/C—H insertion. The good miscibility between iRUM precursors and DPPTT/SEBS significantly improves the covalent crosslinking density in both semiconductor and dielectric layers.

FIG. 1c is a graph illustrating the typical shape of cyclic stress-strain curves of plastic (conventional stretchable) PSC and elastic PSC demonstrated in this work. Conventional stretchable PSC undergoes plastic deformation, with residue strain left after one single loading-unloading cycle. When such a semiconductor film is supported on an elastic dielectric, cyclic strain results in wrinkling and buckling which may lead to interfacial delamination, and significantly degrade charge transport. As shown in FIG. 1d, an iRUM approach according to embodiments achieves the integration of all desired merits for skin-inspired polymer electronics from both daily use and manufacturing perspectives, without compromising electrical performance.

Briefly, for semiconductors, an iRUM precursor consisting of perfluorophenyl azide (PFPA) end-capped polybutadiene, BA (FIG. 1a), was designed to enable the following key features:

(i) The flexible backbone structure and compatible surface energy (30.4 mJ/m²) of BA enabled its good mixing (without forming micrometer-sized large separated domains) with the PSC (30-33 mJ/m²) in a high BA-to-PSC ratio, allowing for high crosslinking density.

(ii) BA was found to undergo self-crosslinking to generate a stretchable and elastic matrix through azide/C═C cycloaddition (Liu, L. H. & Yan, M. Perfluorophenyl azides: New applications in surface functionalization and nanomaterial synthesis. Acc. Chem. Res. 43, 1434-1443 (2010)), resulting in a ‘semiconductor-in-rubber’ composite film.

(iii) The azide groups of BA also reacted with and crosslinked the alkyl side chains on polymer semiconductors through azide/C—H insertion (Peng, R. Q. et al. High-performance polymer semiconducting heterostructure devices by nitrene-mediated photocrosslinking of alkyl side chains. Nat. Mater. 9, 152-158 (2010)) (Supplementary FIG. 1). The created covalent bonding between BA and PSC is necessary for solvent-resistance and photo-patternability.

(iv) A much higher proportion of BA underwent self-crosslinking described in (ii) rather than crosslinking with PSC in (iii), since the reactivity of cyclization is seven times faster than that of azide/C—H insertion (Young, M. J. T. & Platz, M. S. Mechanistic analysis of the reactions of (pentafluorophenyl)nitrene in alkanes. J. Org. Chem. 56, 6403-6406 (1991); Poe, R., Schnapp, K., Young, M. J. T., Grayzar, J. & Platz, M. S. Chemistry and Kinetics of Singlet (Pentafluorophenyl)nitrene. J. Am. Chem. Soc. 114, 5054-5067 (1992)). Therefore, the PSC aggregation was hardly disrupted, resulting in maintained charge transport pathways.

With these features, the PSC network was covalently linked to the as-formed elastomeric matrix (FIG. 1a). Moreover, the hydroxyl-terminated polybutadiene is a widely utilized ingredient in rubber industry for nearly a century and is available in low cost and large scale (Cao, Z., Zhou, Q., Jie, S. & Li, B.-G. High cis-1,4 Hydroxyl-Terminated Polybutadiene-Based Polyurethanes with Extremely Low Glass Transition Temperature and Excellent Mechanical Properties. Ind. Eng. Chem. Res. 55, 1582-1589 (2016)).

For the dielectrics, an iRUM precursor consisting of PFPA end-capped hydrogenated-polybutadiene, BH (FIG. 1b), was designed to crosslink a widely-used stretchable dielectric material, polystyrene-block-poly(ethylene-co-butylene)-block-polystyrene (SEBS). The high crosslinking density of the dielectrics is critical to realize its elasticity, and provides superior solvent resistance which is necessary for direct depositing and photo-patterning of semiconductors on it.

Two important advances were achieved by iRUM and described in more detail below:

(i) Different from conventional stretchable polymer semiconductors, the iRUM approach results in photo-patternable elastic semiconductors with high cyclic reversibility (FIG. 1c) without compromising charge carrier mobility.

(ii) The iRUM approach achieves all desirable merits (high electronic performance, softness, mechanical stability and ease of manufacturing) for polymer electronic materials (FIG. 1d).

iRUM Precursors and Matrix Formation

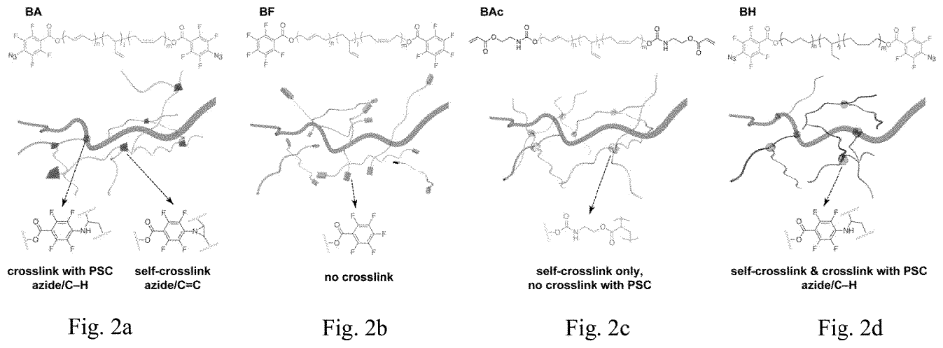

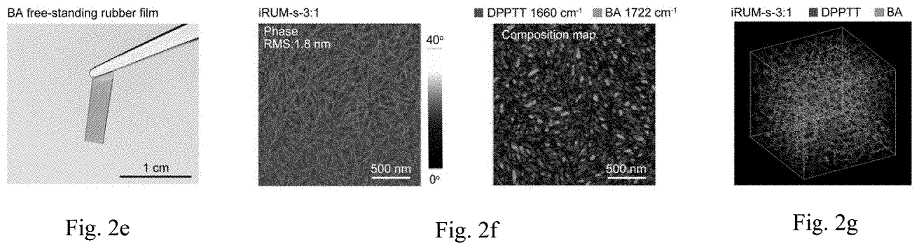

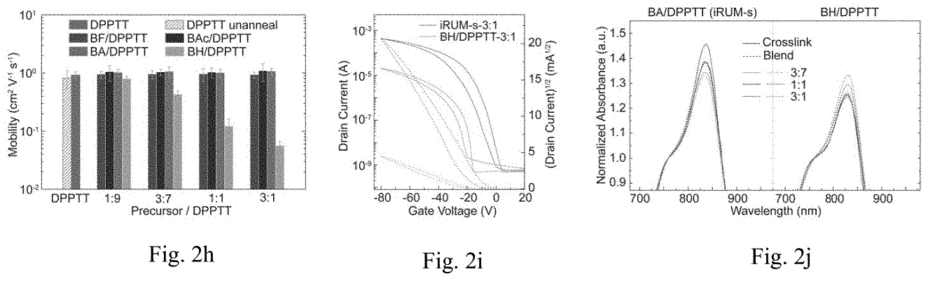

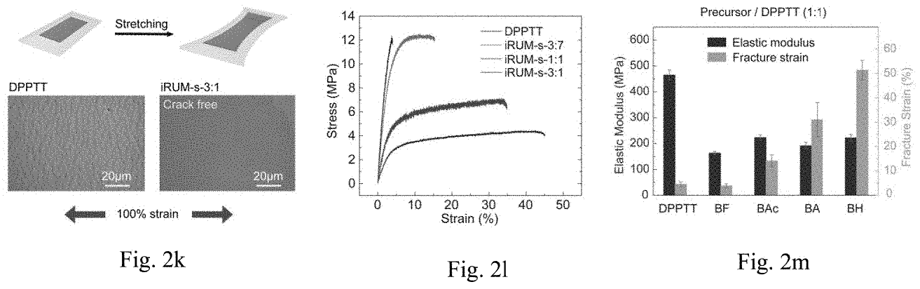

FIG. 2a to 2m illustrate an example systematic investigation of molecular design principles of iRUM semiconductors. The obtained semiconductor film is named ‘iRUM-s-x:y’, in which ‘s’ stands for semiconductor and ‘x:y’ is the BA-to-DPPTT weight ratio. Chemical structures of different polybutadiene-based precursors are shown in FIG. 2a (BA), FIG. 2b (BF), FIG. 2c (Bac), and FIG. 2d (BH), and the generated semiconductor film after blending with DPPTT followed by thermal annealing at 150° C. FIG. 2e illustrates an example free-standing elastic rubber film created by BA through azide/double bond cycloaddition. FIG. 2f provides an example AFM phase image and AFM-IR overlay image of iRUM-s-3:1 film. The surface roughness is obtained from the AFM height image. In the composition map (right), red color represents the DPPTT phase probed by IR laser at 1660 cm⁻¹ and the green color represents the BA phase probed at 1722 cm⁻¹ respectively.

FIG. 2g is a screen shot of MD simulated iRUM-s-3:1 film (simulation box length: —5.8 nm), showing distributions of DPPTT and BA in a blend. FIG. 2h is a bar chart illustrating the extracted mobility of iRUM-s-x:y (BA/DPPTT-x:y) films, BF/DPPTT-x:y blend films, BAc/DPPTT-x:y and BH/DPPTT-x:y crosslinked films where x:y is the precursor-to-DPPTT weight ratio, characterized in bottom-gate top-contact transistors using SiO₂ (300 nm)/Si as dielectric and gate electrode. FIG. 2i provides example representative transfer curves for iRUM-s-3:1 and BH/DPPTT-3:1 films. FIG. 2j provides UV-vis spectrum for iRUM-s and BH/DPPTT films prior and after thermal crosslinking. FIG. 2k provides a schematic illustration of the stretching of an example semiconductor film on a supported PDMS substrate according to embodiments. Optical microscope images of a neat DPPTT film and an iRUM-s-3:1 film under 100% strain. FIG. 2l is a graph providing example stress-strain (engineering) curves for DPPTT and iRUM-s-x:y films obtained from pseudo free-standing tensile tests. FIG. 2m is a graph illustrating the extracted elastic modulus and fracture strain of example iRUM-s (BA/DPPTT) films, BF/DPPTT blend films, BAc/DPPTT and BH/DPPTT crosslinked films according to embodiments obtained from pseudo free-standing tensile tests (the precursor-to-DPPTT weight ratio is 1:1).

The iRUM approach is first illustrating in connection with semiconductors. In order to rationalize the molecular design principles of BA (FIG. 2a), synthesized were different polybutadiene-based precursors:

(i) polybutadiene-fluorine (BF): structurally similar to BA but cannot crosslink (FIG. 2b),

(ii) polybutadiene-acrylate (BAc): can only undergo self-crosslink to form an elastic rubber network, but no covalent crosslink with PSC (FIG. 2c), and

(iii) polybutadiene-hydrogenated-azide (BH): serves as a reference to compare the effect of reactivity difference between azide/C—H insertion and azide/C═C cycloaddition on the semiconductor electrical performance (FIG. 2d).

As described above, it was observed that the iRUM precursor BA can react with itself (self-crosslink) to form an elastic rubber matrix. It showed no residue strain after repeated stretching-releasing cycles (FIG. 2e). Next, chosen were widely-used high mobility donor-acceptor (D-A) conjugated polymers, poly-thieno[3,2-b]thiophene-diketopyrrolopyrrole (DPPTT) (Mn: 62.7 kg/mol, PDI: 2.9), as model semiconductors to investigate the feasibility of our approach. The obtained semiconductor film is named ‘iRUM-s-x:y’, in which ‘s’ stands for semiconductor and ‘x:y’ is the BA-to-DPPTT weight ratio. As observed from atomic force microscopy (AFM), iRUM-s-3:7, -1:1, and -3:1 all exhibited uniform morphology (FIG. 2f) and low surface roughness (1.8-2.4 nm), even though the weight of BA is up to three times that of DPPTT. Additionally, the composition map of the film surface obtained through AFM paired with infrared spectroscopy (AFM-IR) confirmed the well-dispersed DPPTT network within the continuous BA-formed rubber matrix (FIG. 2f). Molecular dynamics (MD) simulations suggest a stronger association between BA and DPPTT side chains than that between BA and DPPTT backbone (FIG. 2g). It is hypothesized that the bi-continuous network formed by BA and polymer semiconductor serves as the key premise in ensuring abundant crosslinking group in composite film and thus high covalent crosslinking density. Similarly, the blended or crosslinked DPPTT films with BF, BAc or BH all exhibited uniform mixing. It is believed these morphologies originate from the surface energy match between the two components (conjugated polymer and polybutadiene-derived precursor), and the high flexibility of the polybutadiene backbone. This flexibility can prevent large-domain (micro-scale) phase separation driven by crystallization of the molecules, as has been observed in other systems.

Maintained semiconductor electrical performance: The electrical performance of iRUM-s-x:y films was characterized in bottom-gate top-contact transistors with highly-doped Si as a gate electrode, MoO₃/Au as source and drain electrodes and octadecyltrimethoxysilane (OTS)-modified SiO₂ as dielectrics. As shown by transfer curves and extracted charge carrier mobilities (FIGS. 2h and 2i), this iRUM approach did not adversely affect the electrical performance of semiconductors, with all mobilities being maintained at ˜1 cm² V⁻¹ s⁻¹ despite the addition of different BA proportions. This mobility value is similar to that of the neat DPPTT. It is hypothesized that this is due to the well-controlled ratio of C═C cycloaddition (i.e. reaction between azide and polybutadiene backbone) versus C—H insertion (i.e. reaction between azide and side chains on polymer semiconductor during crosslinking), as confirmed later. Since the charge carrier mobilities in BF/DPPTT blend films (FIG. 2b) and BAc/DPPTT crosslinked films (FIG. 2c) were also unaffected, it indicates that blending with polybutadiene precursors and the in-situ elastic rubber matrix formed inside the polymer semiconductor network will not disrupt the charge transport pathways (FIG. 2h). On the contrary, the mobility was observed to suffer a drastic decay in BH/DPPTT crosslinked films (0.12 cm² V⁻¹ s⁻¹ for BH/DPPTT-1:1 and 0.06 cm² V⁻¹ s⁻¹ for BH/DPPTT-3:1), as the BH concentration increases (FIGS. 2h and 2i, Supplementary FIG. 16). To better understand this drastic difference, performed were multiple morphological characterizations. As revealed by the depth profiles of X-ray photoelectron spectroscopy (XPS), the semiconductor component in both iRUM-s-1:1 and BH/DPPTT-1:1 crosslinked film showed a similarly uniform distribution across film thickness. Therefore, the difference in electrical performance cannot be simply explained by the different vertical distribution of DPPTTs.

Based on the above observations, it is hypothesized that the difference in reactivity may be contributing. BA may mostly have reacted with its double bonds to create a rubber matrix, instead of reacting with the side chains of semiconductor that will disrupt chain packing and aggregation, thus able to maintain its charge transport pathway. The higher reactivity of azide/C═C cycloaddition than that of azide/C—H insertion is supported by thermogravimetric analysis (TGA) and attenuated total-reflectance Fourier transformation infrared spectroscopy (ATR-FTIR). Different from BA, the all-single-bond backbone on BH is chemically indistinguishable with the long side chains on semiconductors, resulting in their equal reactivity with azides and potentially more disruption of the semiconductor aggregation. This is supported by ultraviolet-visible absorption spectra (UV-vis) results. Specifically, the semiconductor aggregation in BAc/DPPTT and BA/DPPTT (iRUM-s) slightly increased in crosslinked films compared to their simply blended films without crosslinking; while on the contrary, a clear decrease in aggregation was observed in BH/DPPTT films after crosslinking (FIG. 2j). Additionally, as indicated by grazing-incidence X-ray diffraction (GIXD), the edge-on coherence length slightly increased in iRUM-s-x:y films compared with the neat DPPTT film, while the mean coherence length decreased in BH/DPPTT crosslinked films.

In summary, previous semiconductor/small molecule crosslinked systems fell short in simultaneously realizing high covalent crosslinking density (essential for elasticity) and maintained decent charge carrier mobility (Pakhnyuk, V., Onorato, J. W., Steiner, E. J., Cohen, T. A. & Luscombe, C. K. Enhanced miscibility and strain resistance of blended elastomer/it-conjugated polymer composites through side chain functionalization towards stretchable electronics. Polym. Int. 69, 308-316 (2020)). While in the present embodiments, this challenging obstacle was overcome by leveraging the reactivity difference between azide/C—H insertion and azide/C═C cycloaddition, and selecting appropriate rubber precursor structure to induce desired morphology for facilitated charge transport.

Softness and stretchability: Next, the stretchability of iRUM-s-x:y was studied by applying various levels of strain on a polydimethylsiloxane (PDMS)-supported thin film (Lee, J. H., Chung, J. Y. & Stafford, C. M. Effect of confinement on stiffness and fracture of thin amorphous polymer films. ACS Macro Lett. 1, 122-126 (2012)). For iRUM-s-1:1 and -3:1, they can be stretched up to 100% strain without showing any visible cracks. In contrast, neat DPPTT film showed dense and micrometer-scale cracks at 100% strain (FIG. 2k). Furthermore, pseudo free-standing tensile tests were performed by floating the semiconductor film on water, which provided characterization of intrinsic thin-film mechanics (Kim, J.-H. et al. Tensile testing of ultra-thin films on water surface. Nat. Commun. 4, 2520 (2013); Zhang, S. et al. Probing the Viscoelastic Property of Pseudo Free-Standing Conjugated Polymeric Thin Films. Macromol. Rapid Commun. 39, 1800092 (2018)). From the obtained stress-strain curves, the iRUM-s-3:1 film exhibited nearly one-order-of-magnitude increase in fracture strain and four-time decrease in elastic modulus compared to neat film (FIG. 2l). This significant improvement in stretchability and softness originates from the increased proportion of the rubber matrix created by BA, which has a much lower elastic modulus (6.9 MPa) and higher fracture strain (145.9%) than that of pristine DPPTT (464.9 MPa and 4.5%, respectively). When such an iRUM-s film undergoes mechanical deformation, the in-situ-formed crosslinked matrix provides stretchability and elasticity. Without crosslinking, the blend film of BF/DPPTT exhibited a much lower elastic modulus than neat DPPTT film, which was attributed to a plasticizing effect caused by the non-crosslinked polybutadiene (Selivanova, M. et al. Branched Polyethylene as a Plasticizing Additive to Modulate the Mechanical Properties of it-Conjugated Polymers. Macromolecules 52, 7870-7877 (2019)). However, its fracture strain is still as low as 3.9% and cracks still formed at 50% strain despite reduced crack sizes. The other two types of semiconductor films with crosslinked matrices, BAc/DPPTT-1:1 and BH/DPPTT-1:1, also showed improved softness and stretchability than the neat film (FIG. 2m).

### Elastic and Stretchable Transistors

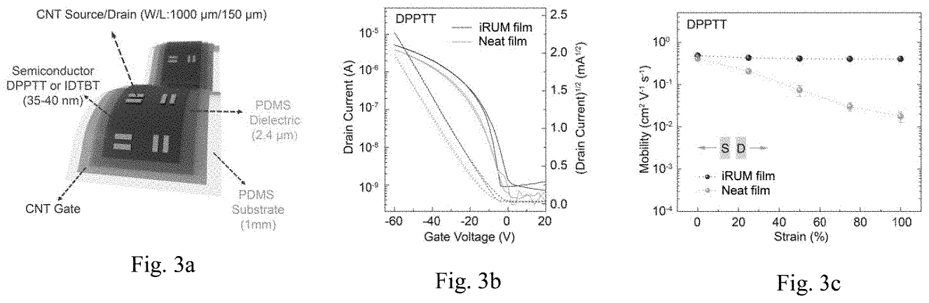

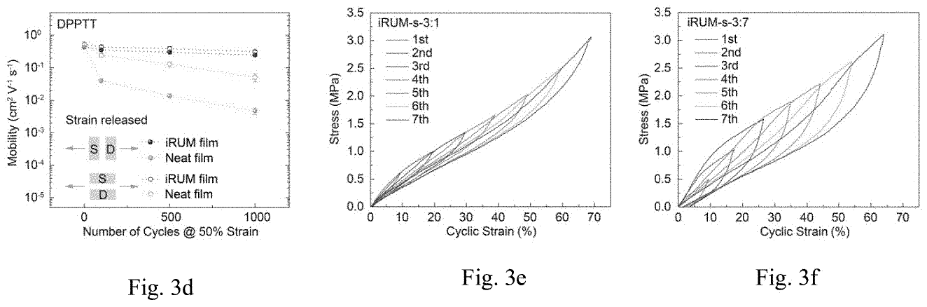

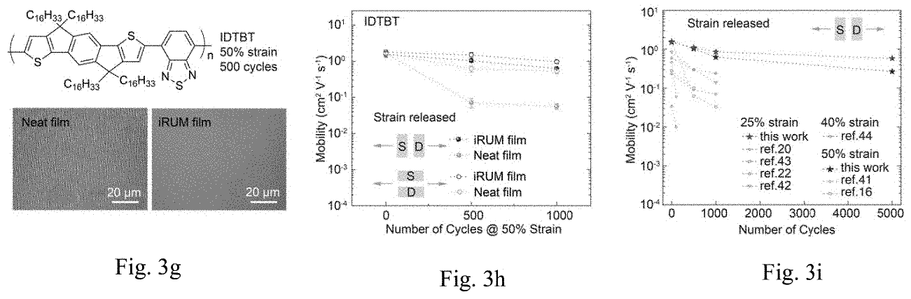

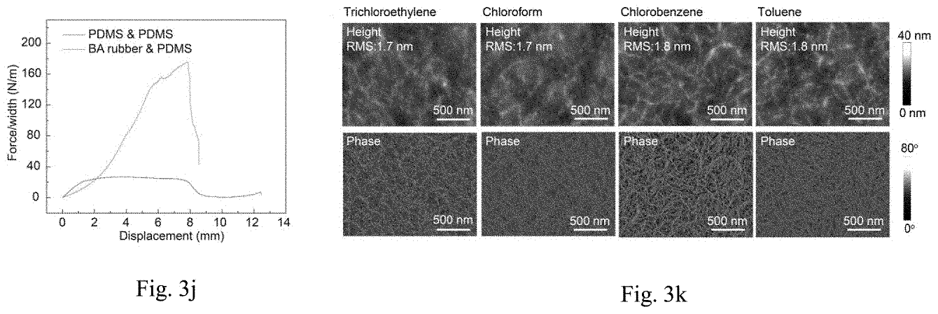

FIG. 3a to 3k illustrate an example characterization of the electrical performance of iRUM-s in accordance with embodiments under mechanical deformation and solvent resistance. FIG. 3a is a block diagram illustrating a device structure of an example fully stretchable transistor with a bottom-gate top-contact configuration. In this example, W/L=1000 μm/150 μm. FIG. 3b is a graph providing example transfer curves of an example stretchable transistor with neat DPPTT film or iRUM-s as the semiconductor. FIG. 3c is a graph illustrating evolution in mobility at different strains during single stretching, with charge transport parallel to stretching direction. FIG. 3d is a graph illustrating evolution in mobility after multiple stretching-releasing cycles at 50% strain under strain released state, with charge transport parallel and perpendicular to stretching direction. FIGS. 3e and 3f are stress-strain (engineering) curves with a cyclic strain range of 10-70% for iRUM-s-3:1 and iRUM-s-3:7 films in accordance with embodiments, respectively, where an iRUM-s film (35 nm thick) was supported on a thin PDMS substrate (2.4 μm thick).

FIG. 3g provides optical microscope images of example IDTBT and iRUM-IDTBT according to embodiments after 500 stretching-releasing cycles at 50% strain under strain released state. FIG. 3h is a graph illustrating changes in mobility of a neat IDTBT film and an iRUM-IDTBT film according to embodiments after multiple stretching-releasing cycles at 50% strain under strain released state. FIG. 3i provides a comparison of the cyclic durability in the present embodiments with previously reported results in the literature (spin-coated semiconductor films using insulating polymer dielectrics under strain released state without applying other engineering techniques, which reflects the intrinsic properties of semiconductors). FIG. 3j provides curves of the peeling force per width of PDMS sheet versus displacement for BA rubber and PDMS. The higher peeling force between BA rubber and PDMS indicates the formation of interfacial crosslinking. FIG. 3k are AFM height and phase images of iRUM-s-3:1 after soaking in various organic solvents (trichloroethylene, chloroform, chlorobenzene and toluene) for 30 s. The surface roughness is extracted from the AFM height image.

As shown in the above figures, stretchable transistors were fabricated to evaluate the electrical performance of iRUM-s under mechanical deformation, with carbon nanotube (CNT) as gate and source/drain electrodes and PDMS as dielectrics (FIG. 3a). As shown in a representative transfer curve, iRUM-s film showed ideal and comparative transistor performance as neat film, with an average mobility of 0.5 cm² V⁻¹ s⁻¹ (FIG. 3b). The decreased mobility compared to the rigid transistors originates from higher contact resistance of CNT than Au electrode (Zheng, Y. et al. Tuning the Mechanical Properties of a Polymer Semiconductor by Modulating Hydrogen Bonding Interactions. Chem. Mater. 32, 5700-5714 (2020)). The mobility of iRUM-s was stably maintained despite the device being stretched up to 100% strain along the charge transport direction, while the neat film exhibited dramatic mobility degradation (FIG. 3c). As pointed out before, practical electronic devices need to reliably operate beyond a single stretching and maintain its functionalities under harsh cyclic loading. Therefore, performed were cyclic tests on the transistor at 50% strain (higher than typically applied strains needed for skin-inspired electronics, i.e. ˜20-30%). The iRUM-s film showed remarkably stable charge carrier mobility (0.3 cm² V¹ s⁻¹) and on-current after 1000 stretching-releasing cycles under strain released state (FIG. 3d). This excellent cyclic durability was attributed to the robust elasticity of composite semiconductor film enabled by the iRUM approach of embodiments.

To confirm the obtained elasticity improvement, next performed were mechanical tests for a bilayer specimen where an iRUM-s film (35 nm thick) was supported on a thin PDMS substrate (2.4 μm thick). This condition reflects the mechanical response of a semiconductor in a practical device configuration. A recent report demonstrated the feasibility of this film-laminated-on-thin-elastomer (FLOTE) approach to measure the viscoelastic behavior of a conjugated polymer thin film under large cyclic strains (Song, R. et al. Unveiling the Stress—Strain Behavior of Conjugated Polymer Thin Films for Stretchable Device Applications. Macromolecules 53, 1988-1997 (2020)). The viscoelasticity of conjugated polymer film was then extracted from the measurement on the bilayer structure and was found to significantly impact film stability after cycling. For our iRUM-s-3:1 film which was used in stretchable transistors, the stress-strain curves with increasing cyclic strains from 10% to 70% showed no residual strain upon strain removal (FIG. 3e). By contrast, the iRUM-s-3:7 film exhibited a ˜4% residue strain due to lower rubber matrix content and thus plastic deformation (FIG. 3f), which is consistent with the observation of wrinkle formation after repeated stretching cycles observed via AFM. Furthermore, iRUM-s-3:1 film showed less stress relaxation and a much-reduced hysteresis, indicating the transition from viscoelasticity to entropy-driven elastic/reversible behavior (Sheiko, S. S. & Dobrynin, A. V. Architectural Code for Rubber Elasticity: From Supersoft to Superfirm Materials. Macromolecules 52, 7531-7546 (2019)). When such a semiconductor film is deformed, the strain energy can be dissipated through conformational change of the in-situ formed rubber matrix, while the covalent crosslinking sites provide the film with ‘rebound force’ to return to its original state. The underlying mechanism was further cross-validated by the existence of residue strain and higher hysteresis as obtained from cyclic tests of BF/DPPTT-3:1 blend film and BAc/DPPTT-3:1 crosslinked film. This provides a direct demonstration of the elastic behavior of polymer semiconductor film under cyclic mechanical testing.

To examine the versatility of an example iRUM approach according to embodiments, it was applied to another high-mobility D-A conjugated polymer, indacenodithiophene-co-benzothiadiazole (IDTBT) (Venkateshvaran, D. et al. Approaching disorder-free transport in high-mobility conjugated polymers. Nature 515, 384-388 (2014)) (Mn: 104.5 kg/mol, PDI: 2.8). Different from semicrystalline DPPTT, IDTBT is known to exhibit a quasi-amorphous morphology and low energetic disorder. It was previously reported that IDTBT exhibited high stretchability (crack on-set strain >100% when supported on a PDMS substrate) but poor cyclic durability due to plastic deformation, as confirmed by clear wrinkle formation and sharp mobility decrease (from >1 down to 0.07 cm² V⁻¹ s⁻¹) after 500 stretching-releasing cycles at 50% strain (FIGS. 3g and h). Currently, no approach has been reported to transform such a plastic semiconductor to an elastic one. After applying iRUM approach on IDTBT, the previously observed wrinkle formation for neat IDTBT after repeated stretching cycles was completely eliminated. Micrometer-sized large separated domains were not observed in the composite film, confirming the good mixing between BA and IDTBT. The average mobility was maintained at >1 cm² V⁻¹ s⁻¹ even after 500 loading-unloading cycles at 50% strain under strain released state in fully stretchable transistors (FIGS. 3g and 3h). This is already a higher mobility retention compared to all other strategies reported in the literature thus far, and the applied cyclic strain level (50%) is two times higher than conventional strain level (Mun, J. et al. Effect of Nonconjugated Spacers on Mechanical Properties of Semiconducting Polymers for Stretchable Transistors. Adv. Funct. Mater. 28, 1804222 (2018); Mun, J. et al. Conjugated Carbon Cyclic Nanorings as Additives for Intrinsically Stretchable Semiconducting Polymers. Adv. Mater. 31, 1903912 (2019); Oh, J. Y. et al. Intrinsically stretchable and healable semiconducting polymer for organic transistors. Nature 539, 411-415 (2016); Shin, M. et al. Polythiophene nanofibril bundles surface-embedded in elastomer: A route to a highly stretchable active channel layer. Adv. Mater. 27, 1255-1261 (2015)) (FIG. 3i). The cycling life of an example iRUM-s film of embodiments can even be stably extended to 5000 cycles (four-hour continuous stretching-releasing), which is over five times longer than any previous attempt (FIG. 3i). As a comparison, observed was poor cyclic durability for non-crosslinked BF/IDTBT blend film, thus confirming again the elastic property of iRUM-IDTBT is a critical factor for the stable cycling performance rather than solely modulus match or interfacial crosslinking with dielectrics. Furthermore, it observed that the stretchability of iRUM-IDTBT film was maintained while its softness was increased (reduced elastic modulus), as confirmed by pseudo free-standing tensile tests and crack on-set strain characterizations.

Interfacial crosslinking: The accessible C═C double bonds of BA backbone located on the surfaces of iRUM-s film provide additional opportunities to engineer the semiconductor-dielectric interface. These double bonds are able to undergo various chemical reactions, e.g. hydrosilylation with silicon-hydride (Si—H), thiol-ene reaction with S—H group (S y Piecco, K. W. E. et al. Reusable Chemically Micropatterned Substrates via Sequential Photoinitiated Thiol—Ene Reactions as a Template for Perovskite Thin-Film Microarrays. ACS Appl. Electron. Mater. 1, 2279-2286 (2019)), and cycloaddition with azide. It was reasoned that interfacial crosslinking can be created between BA rubber and PDMS through Si—H/vinyl reactions during curing process, as evidenced by a much higher interfacial toughness obtained from 180o peeling tests when compared to PDMS/PDMS interface (Yuk, H., Zhang, T., Lin, S., Parada, G. A. & Zhao, X. Tough bonding of hydrogels to diverse non-porous surfaces. Nat. Mater. 15, 190-196 (2016)) (FIG. 3j). It was observed that the PDMS portion fractured even before the breakage of BA/PDMS interface, while two pieces of PDMS can be readily separated. Such a strong interfacial interaction ensures good adhesion and prevents delamination between semiconductor and dielectric, which is beneficial to stable operation of electronic devices under cyclic mechanical deformation over a long period of time.

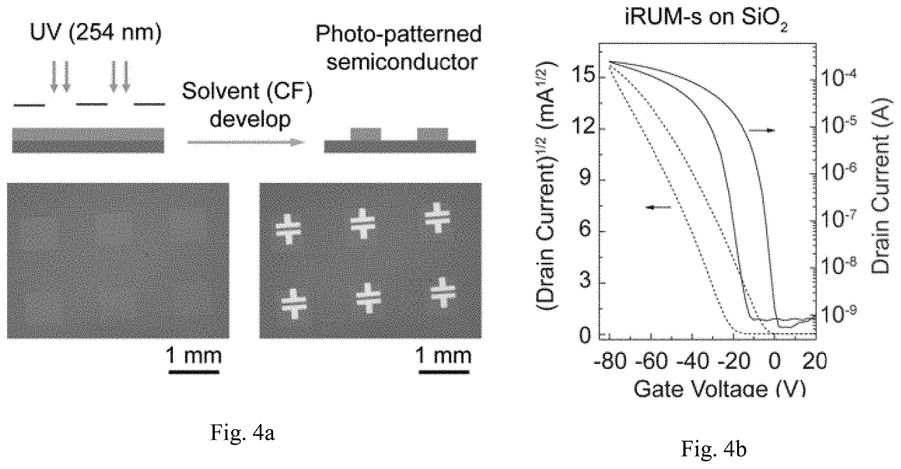

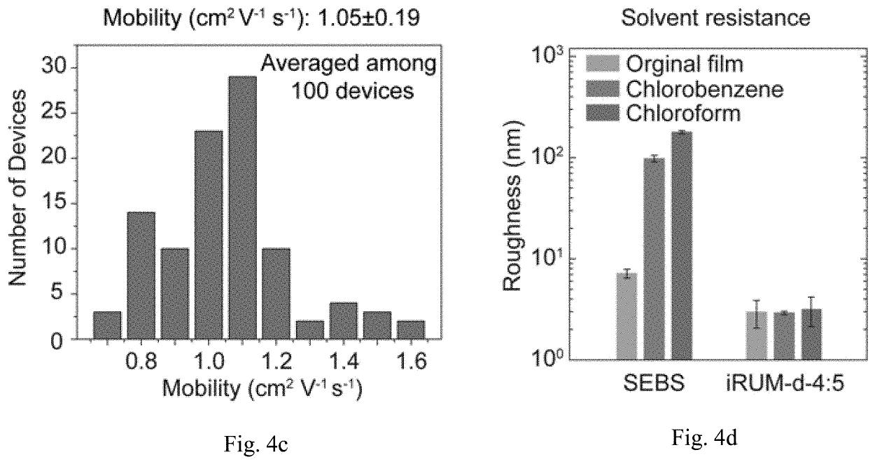

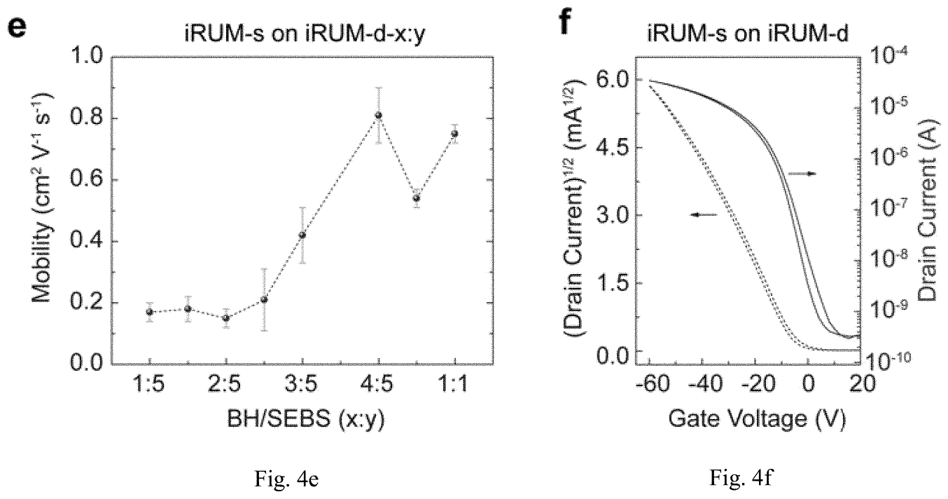

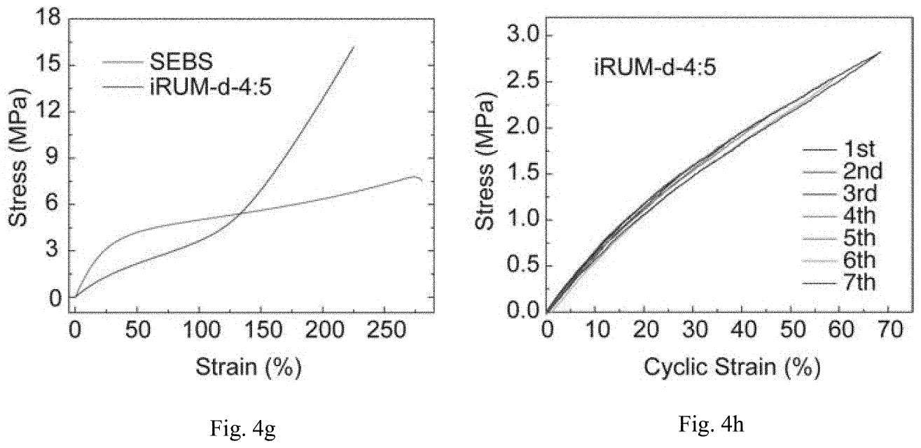

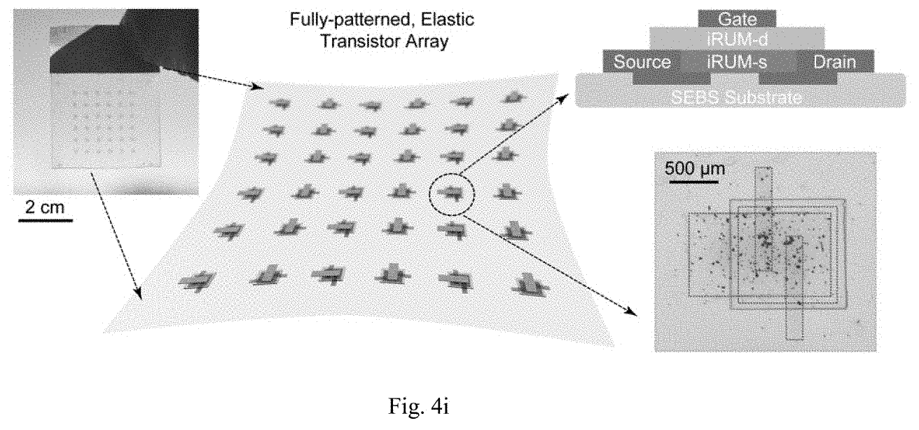

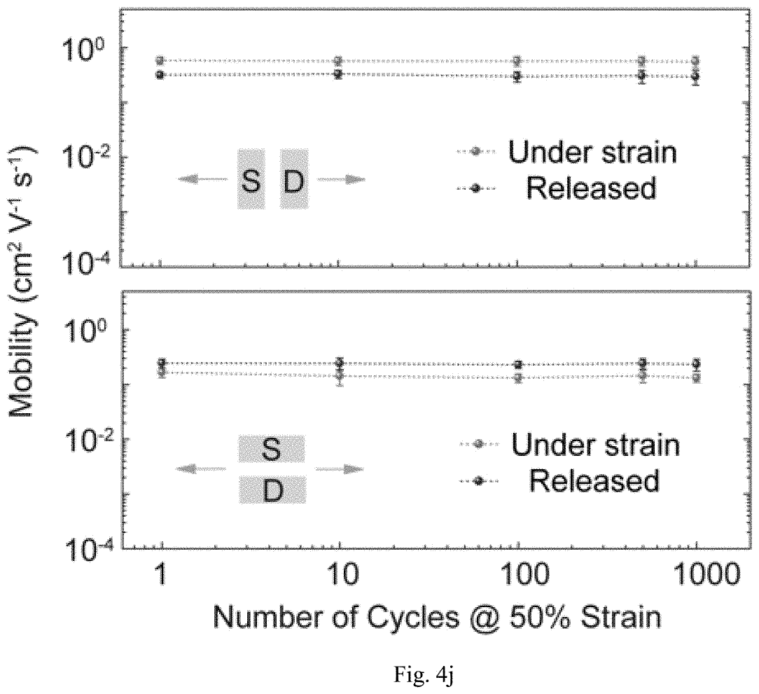

Solvent resistance and photo-patternability: The chemical crosslinking nature of the iRUM approach provides solvent resistance for polymer semiconductors, which is necessary for multilayer device fabrication. After treating a crosslinked iRUM-semiconductor film with various organic solvents, such as trichloroethylene, chloroform, chlorobenzene and toluene which are commonly used to process polymer semiconductors, the surface remained smooth with low roughness (˜1.7 to 1.8 nm) indicating little material being dissolved away by solvent. In addition, the morphology was well-maintained as observed from both height and phase images by AFM (FIG. 3k). This observation was attributed to the covalent bonding between the in-situ formed BA rubber and polymer semiconductor network. On the other hand, BAc/DPPTT crosslinked film and BF/DPPTT blend film were found to be either partially or totally removed after organic solvent treatment. Therefore, the covalent bonding between BA and DPPTT created through C—H insertion is necessary for solvent resistance and photo-patternability in addition to C═C cycloaddition of the BA matrix. Besides solvent resistance, it was reasoned that since the crosslinking reaction of azide group can be activated by UV light, an iRUM approach according to embodiments can be used to photo-pattern polymer semiconductors. Through selective UV exposure (254 nm UV for 1 min), the photo-crosslinked regions of iRUM-s film will be immobilized, while the non-crosslinked regions can subsequently be ‘washed away’ using appropriate organic solvents (FIG. 4a). More importantly, no degradation was observed in electrical performance of the iRUM-s film during photo-crosslinking and chloroform solvent development process. As a result, the photo-patterned semiconductor film exhibited equivalently high mobility compared with neat film, achieving an averaged mobility of 1.05 cm² V⁻¹ s⁻¹ for 10×10 photo-patterned transistors on SiO₂/Si substrate (FIGS. 4b and 4c).

Summary of Example iRUM-Semiconductor

FIGS. 4a to 4j illustrate example aspects of the photo-patternability of iRUM-s and iRUM-d according to embodiments, and the integrated patterned elastic transistors on a single substrate. FIG. 4a is a schematic illustration of an example photo-patterning process for iRUM-s, which involves selective UV exposure and chloroform development. Optical microscope images of photo-patterned iRUM-s film (thickness: 35 nm) before and after depositing MoO3/Au (2 nm/55 nm) electrodes are provided. FIG. 4b is a graph providing an example transfer curve of iRUM-s film that is photo-patterned on OTS-modified SiO₂ (300 nm) as characterized in a bottom-gate top-contact transistor. FIG. 4c is a graph providing an example mobility distribution of photo-patterned iRUM-s according to embodiments from 10×10 photo-patterned transistors on a single substrate, with bottom-gate top-contact configuration, highly-doped Si as gate electrode, MoO₃/Au as source/drain electrodes (W=500 μmm, L=50 μm) and octadecyltrimethoxysilane (OTS)-modified SiO2 (300 nm) as dielectrics. FIG. 4d is a graph illustrating an example surface roughness characterization of SEBS and iRUM-d-4:5 films by profilometer before- and after-solvent treatment. FIG. 4e is a graph illustrating the mobility of an example photo-patterned iRUM-s film on iRUM-d-x:y (1-1.5 μm thick), where x:y is the BH-to-SEBS weight ratio. FIG. 4f is a graph providing an example transfer curve of a bottom-gate top-contact transistor with iRUM-s according to embodiments directly photo-patterned on iRUM-d-4:5 (1 μm thick). FIG. 4g is a graph providing representative stress-strain curves for SEBS and iRUM-d-4:5 films according to embodiments obtained from pseudo free-standing tensile tests. FIG. 4h are graphs providing example cyclic stress-strain curves (10-70% strains) for iRUM-d-4:5 according to embodiments. FIG. 4i is a schematic of fully patterned, stretchable and elastic transistors on a single substrate according to embodiments. Two-dimensional diagram showing the side view of the transistor structure. FIG. 4j is a graph illustrating changes in mobility for the example patterned elastic transistors after multiple stretching-releasing cycles at 50% strain, with charge transport parallel and perpendicular to stretching direction.

As shown in the above figures, the example results underscore the following molecular design guidelines towards multifunctional PSC: (i) Uniform mixing of rubber matrix precursors with conjugated polymers is the premise of high covalent crosslinking density. (ii) Stretchability and even elasticity can be achieved through the as-formed crosslinked rubber matrix. (iii) Covalent crosslinking between precursor and PSC is necessary for solvent resistance and facile photo-patternability. (iv) The composite film morphology needs to be finely controlled through balancing the process of forming a rubber matrix and crosslinking PSC, in order to maintain charge transport. In addition to the polybutadiene backbone and crosslinkable azide end-group demonstrated in this study, it is believed that other rubber matrix precursors can potentially be adopted as long as they meet with the above requirements.

iRUM-polymer dielectrics: Besides polymer semiconductors, one can further apply the present iRUM approach to a high-performance dielectric material, SEB S. Direct solution-based deposition of semiconductors on top of dielectrics is an industry-friendly process yet long-standing challenge in this field, so one should test the iRUM strategy to address the above limitation. BH was chosen as the precursor to maximize the number of covalent crosslinking sites with SEBS (as elaborated above, BA tends to react more with itself while BH has relatively equal reactivity with both SEBS and itself) and to avoid current-voltage hysteresis that may arise from C═C as traps in the polybutadiene backbone (Wang, B. et al. High-k Gate Dielectrics for Emerging Flexible and Stretchable Electronics. Chem. Rev. 118, 5690-5754 (2018); Wang, Y. et al. Polymer-Based Gate Dielectrics for Organic Field-Effect Transistors. Chem. Mater. 31, 2212-2240 (2019)). The obtained dielectric is named ‘iRUM-d-x:y’, where ‘d.’ stands for dielectric and ‘x:y’ is the BH-to-SEBS weight ratio. BH exhibited excellent mixing with SEBS, without large-domain phase separation occurring until BH/SEBS=1:1. The iRUM-d film was measured to have a similar dielectric constant as SEBS (2.1-2.2). To examine the solvent resistance of iRUM-d films, surface roughness characterization by profilometer before and after solvent treatment was conducted (FIG. 4d). iRUM-d-4:5 film remained smooth and uniform after depositing chlorobenzene or chloroform, with almost no change in roughness (˜3 nm). In contrast, pristine SEBS film showed substantial swelling after solvent treatment, with roughness increased from 7 nm to 178 nm. iRUM-d films with lower crosslinking density showed less swelling but still increased surface roughness after solvent washing. Therefore, increasing the covalent crosslinking density in dielectrics resulted in its improved resistance against organic solvent attack.

Next, fabricated were bottom-gate top-contact transistors with iRUM-s directly deposited and photo-patterned on top of iRUM-d (highly doped Si as gate and MoO₃/Au as source and drain electrodes). It was observed that increasing the amount of BH in the crosslinked dielectric resulted in an increased charge carrier mobility of photo-patterned iRUM-s, with an averaged value of 0.8 cm² V⁻¹ s⁻¹ when using iRUM-d-4:5, and the transistor exhibited ideal transfer characteristics with low hysteresis (FIGS. 4e and 4f). This condition was harsher than that encountered during simple spin-coating or inkjet-printing process using semiconductor solution, as the materials need to survive multiple solvent washing steps. Notably, no further surface modifications on iRUM-d were needed to achieve the desired uniform iRUM-s film. Additionally, despite significantly increased crosslinking density of SEBS, the iRUM-d film showed negligible changes in fracture strain and, in fact, a 50% decrease in elastic modulus. As observed from stress-strain curves (FIGS. 4g and 4h), iRUM-d (1.2 μm thick) exhibited strain-hardening behavior and reduced hysteresis from cyclic mechanical testing as compared to pristine SEBS, underscoring the iRUM-d superior elasticity.

Fully-patterned elastic transistors: Finally, iRUM-d can be successfully photo-patterned by UV (254 nm) exposure and solvent development. Accordingly, iRUM-s and iRUM-d can be incorporated into patterned elastic transistors on a single substrate, which serves as the building-block elements in functional circuits for signal processing and computation (Dai, Y., Hu, H., Wang, M., Xu, J. & Wang, S. Stretchable transistors and functional circuits for human-integrated electronics. Nat. Electron. 4, 17-29 (2021)), thus demonstrating the feasibility of integrating these newly developed materials and producing realistic skin-inspired electronics (FIG. 4i). Specifically, iRUM-d film was first photo-patterned on top of a water-soluble sacrificial layer poly(sodium-4-styrene sulfonate) (PSSNa) (Ji, D., Donner, A. D., Wilde, G., Hu, W. & Fuchs, H. Poly(sodium-4-styrene sulfonate) (PSSNa)-assisted transferable flexible, top-contact high-resolution free-standing organic field-effect transistors. RSC Adv. 5, 98288-98292 (2015)). Next, iRUM-s was directly spin-coated and photo-patterned on the top of iRUM-d, greatly simplifying the conventional protection-etching process for patterning semiconductors. This takes not only the advantages of the photo-patternability of iRUM-s, but also the chemical robustness of iRUM-d, which makes it compatible with layer-by-layer solution deposition during device fabrication. After patterning the CNT source and drain electrodes, laminating substrate, releasing device in water, and patterning CNT gate electrodes, we obtained an average charge carrier mobility of 0.4 cm² V⁻¹ s⁻¹ and a high mobility retention after 1000 stretching-releasing cycles at 50% strain (FIG. 4j).

Generic Description

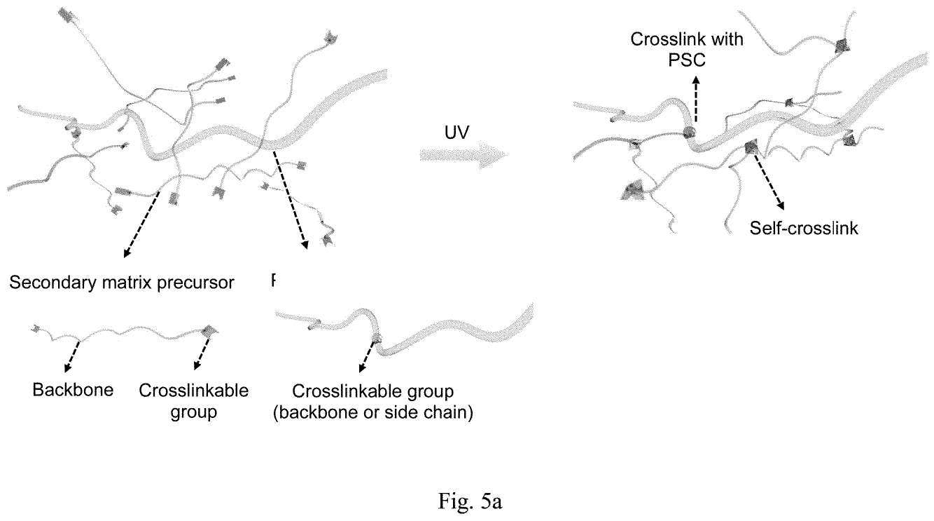

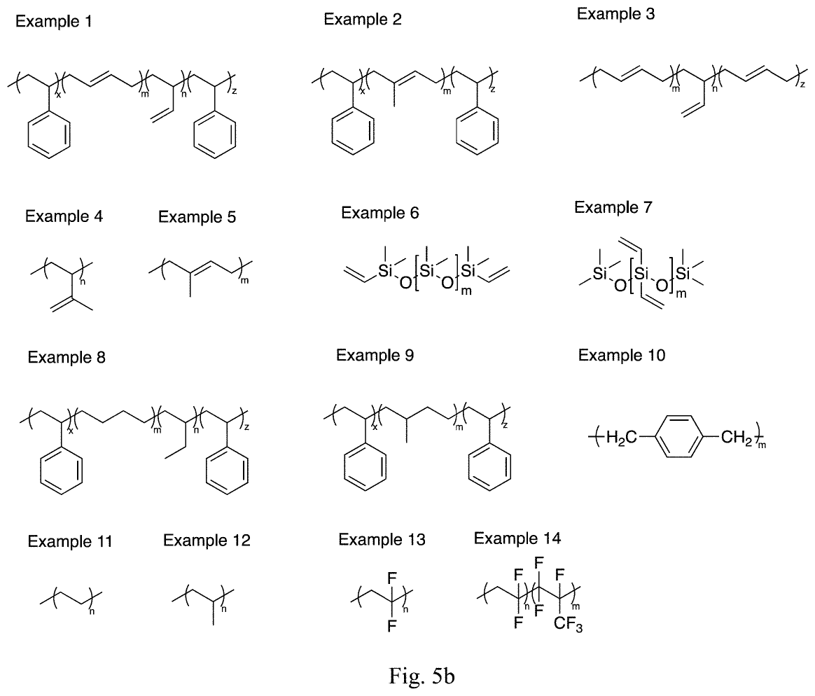

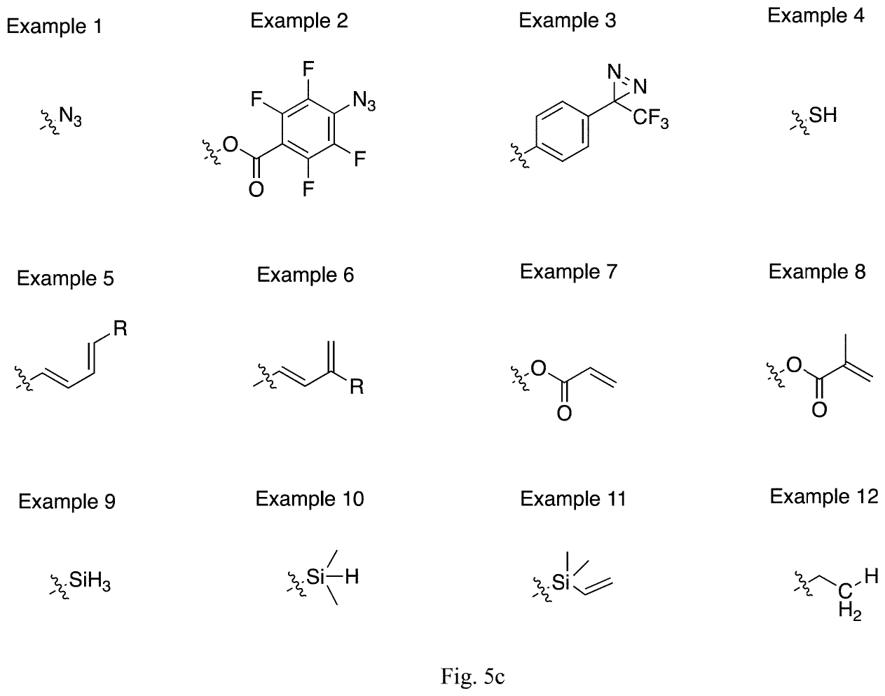

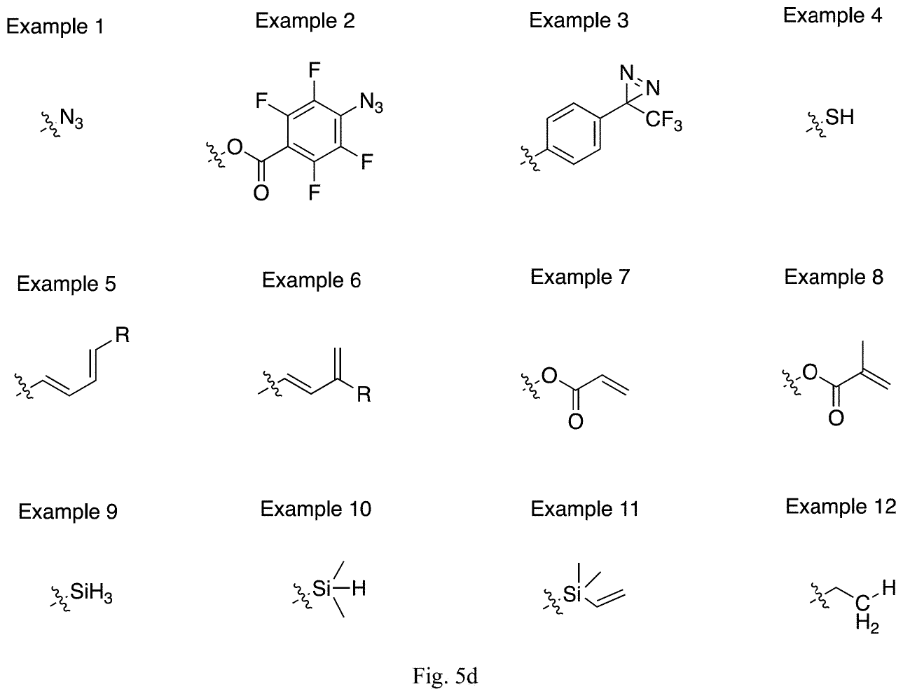

The present embodiments include a molecular design concept that can simultaneously achieve elasticity, high electrical performance, solvent-resistance and photo-patternability in polymer semiconductors and dielectrics. As shown in FIG. 5a, this is enabled by the rational design of a secondary rubber matrix precursor, which can undergo self-crosslink upon UV irradiation after blending with polymer semiconductors. The crosslinkable group can be attached on the chain end or backbone of a precursor. While at the same time, the precursor can react with the crosslinkable groups on polymer semiconductors (either backbone or side-chain). These two crosslinking processes happen simultaneously and the relative ratio can be finely controlled. A higher proportion of precursor undergoes self-crosslink rather than crosslinking with polymer semiconductors.

Potential crosslinking reactions include: (1) Azide/C—H insertion. (2) Azide/C═C cycloaddition. (3) Thiol-ene. (4) Radical polymerization. (5) Diels-Alder reaction. (6) Diazarine/C—H. (7) Hydrosilylation.

FIG. 5b illustrate example secondary matrix backbone structures: (1) poly(styrene-butadiene-styrene), (2) poly(styrene-isoprene-styrene), (3) polybutadiene, (4) (5) polyisoprene (natural rubber), (6) (7) polydimethylsiloxane (PDMS), (8) poly(styrene-ethylene-butylene-styrene), (9) poly(styrene-isobutylene-styrene), (10) parylene, (11) polyethylene, (12) polypropylene, (13) polyvinylidene fluoride (PVDF), (14) poly(vinylidene fluoride)-co-hexafluoropropylene (PVDF-HFP).

Molecular weight range (Mn): 1 k-100 k

FIG. 5c illustrates example secondary matrix crosslinkable group structures.

FIG. 5d illustrates structures of example crosslinkable groups on PSCs. Molecular weight range (Mₙ) of polymer semiconductors: 15 k-150 k. Polymer semiconductors include donor-acceptor polymers, polythiophene and polyacetylene.

Discussion

In summary, the above descriptions demonstrate that covalently-embedded in-situ rubber matrix (iRUM) formation according to embodiments is a simple and effective molecular design approach to simultaneously achieve mechanical robustness, photo-patternability and high electrical performance for polymer electronic materials. On aspect is the good mixing combined with finely controlled composite film morphology built on azide chemistry, which takes advantage of its different reactivities with C—H and C═C bonds. The present iRUM approach results in an elastic, long-cycling semiconductor whose charge carrier mobility is comparable to that of amorphous Si and further increase is possible with other recently reported high mobility polymers. Additionally, the developed materials are compatible with solution-processed multilayer device fabrication and can be incorporated into future complex integrated circuits. The iRUM strategy is also promising for mass production when considering the cost-effectiveness and scalability of precursors as well as the reduced cost of expensive active materials resulting from the high content of iRUM precursors (˜50%-75%). In addition to the transistors demonstrated herein, the vertical uniformity of iRUM-semiconductor film opens up potential applications in vertical electronic devices such as organic light-emitting diodes and organic photovoltaics. The highly accessible and reactive double bonds in iRUM films further provide unique opportunities for pre- and/or post-modification and interfacial engineering through chemical functionalization. The present embodiments represent a milestone in molecular-level design for the transition from soft/stretchable to elastic and multifunctional skin-inspired electronics.

Commercial Applications of the present embodiments include Stretchable electronic devices such as stretchable transistors, flexible LED screen, soft sensors, etc. Industry that is focused on soft and flexible electronics will be interested in this invention, e.g. Samsung.

Advantages and improvements over existing methods, devices or materials: the present embodiments constitute an important milestone in molecular-level design for mechanically robust and multifunctional organic electronic materials. For the first time, we achieve an intrinsically elastic polymer semiconductor while it concurrently possesses all the desired merits in this field, including elasticity, softness, stretchability, solvent-resistance and photo-patternability, without comprising its electrical performance. This work can serve as a beacon that points out the path towards materials for realistic skin-inspired electronics. When applied in fully stretchable transistors, the iRUM-semiconductor film retains its mobility after stretching to 100% strain, and exhibits 1 cm² V⁻¹ s⁻¹ record-high mobility retention after 1000 stretching-releasing cycles at 50% strain. The cycling life is stably extended to 5000 cycles, five times longer than all previous attempts.

Furthermore, fabricated was a fully-patterned elastic transistor array via consecutively photopatterning of dielectrics and semiconductors, demonstrating the feasibility of integrated solution-processed electronics manufacturing. The iRUM approach represents an important advance in molecule-level design for robust and multifunctional skin-inspired electronics.

Since the present materials are already low cost and scalable, find potential industrial partners and their application fields can be the next steps.

The attached Appendix, including Supplemental Information and Supplemental Figures, forms part of the present disclosure and is incorporated herein by reference.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

What is claimed is:

1. A method for obtaining polymer semiconductors or dielectrics, comprising:
preparing a secondary rubber matrix precursor, which can undergo self-crosslink upon UV irradiation after blending with polymer semiconductors or dielectrics, including a crosslinkable group that can be attached on the chain end or backbone of a precursor.

2. The method of claim 1, wherein the precursor is configured to react with the crosslinkable groups on the polymer semiconductors or dielectrics.

3. The method of claim 2, wherein the two crosslinking processes happen simultaneously and the relative ratio can be finely controlled.

4. The method of claim 2, wherein crosslinking reactions include one or more of: Azide/C—H insertion, Azide/C═C cycloaddition, Thiol-ene, Radical polymerization, Diels-Alder reaction, Diazarine/C—H, Hydrosilylation, non-covalent crosslinking.

5. The method of claim 4, wherein the crosslinking reactions can be initiated by one or both of heat and light.

6. The method of claim 1, wherein secondary matrix backbone structures include one or more of: poly(styrene-butadiene-styrene), poly(styrene-isoprene-styrene), polybutadiene, polyisoprene (natural rubber), polydimethylsiloxane (PDMS), poly(styrene-ethylene-butylene-styrene), poly(styrene-isobutylene-styrene), parylene, polyethylene, polypropylene, polyvinylidene fluoride (PVDF), and poly(vinylidene fluoride)-co-hexafluoropropylene (PVDF-HFP).

7. The method of claim 1, wherein a molecular weight range (Mₙ) of the polymer semiconductors is about 15 k to about 150 k.

8. The method of claim 1, wherein the polymer semiconductors include donor-acceptor polymers, poly (naphthalene diimide) derivative, polythiophene and polyacetylene, and wherein the polymer semiconductors can be either p-type or n-type.

9. The method of claim 1, wherein the secondary matrix is formed by covalent crosslinking while with polymer semiconductors or dielectrics are formed through one of covalent or non-covalent crosslinking.

10. A method of forming a covalently-embedded in-situ rubber matrix (iRUM), comprising:
mixing combined with finely controlled composite film morphology built on azide crosslinking chemistry,
wherein the mixing combined with finely controlled composite film morphology built on azide crosslinking chemistry takes advantage of different reactivities with C—H and C═C bonds.

11. The method of claim 10, further comprising deriving a dielectric from the iRUM formation.

12. The method of claim 10, further comprising deriving a semiconductor layer from the iRUM formation.

13. The method of claim 10, further comprising photo-patterning dielectric and semiconducting layers from the iRUM formation.

14. The method of claim 13, further comprising fabricating a stretchable transistor from the dielectric and semiconducting layers.

15. A material for semiconductors or dielectrics comprising:
a covalently-embedded in-situ rubber matrix (iRUM); and
a polymer semiconductor.

16. The material of claim 15, wherein the iRUM is synthesized from a polybutadiene-based precursor.

17. The material of claim 16, wherein the precursor comprises polybutadiene-fluorine (BF).

18. The material of claim 16, wherein the precursor comprises polybutadiene-acrylate (BAc).

19. The material of claim 16, wherein the precursor comprises polybutadiene-hydrogenated-azide (BH).

20. The material of claim 15, wherein the polymer semiconductor comprises a high mobility donor-acceptor (D-A) conjugated polymers, poly-thieno[3,2-b]thiophene-diketopyrrolopyrrole (DPPTT).
